(19) United States
(12) Patent Application Publication
(10) Pub. No.: US 2023/0099708 A1
(43) Pub. Date: Mar. 30, 2023

(54) AUTOMATED FACILITATOR FOR COMMUNITY QUESTION-ANSWER PROCESSING

(71) Applicant: Amicus Brain Innovations, Inc., Chappaqua, NY (US)

(72) Inventors: Chitra Dorai, Chappaqua, NY (US); Edith Helen Stern, Yorktown Heights, NY (US)

(21) Appl. No.: 17/486,806

(22) Filed: Sep. 27, 2021

(51) Int. Cl. G06F 16/332; H04L 12/58; G06F 40/279; G06F 40/30; G06F 16/33; G06F 16/955

(52) U.S. Cl. CPC G06F 16/3329; H04L 51/046; H04L 51/16; G06F 40/279; G06F 40/30; G06F 16/3344; G06F 16/9558

(57) ABSTRACT

The automated third-party facilitation in a community chat includes monitoring an exchange of an inquisitor post in a community chat and an expected response to the inquisitor post. The expected response is then processed in order to compute a sufficiency score measuring a sufficiency of the expected response to a question present in the inquisitor post. Thereafter, on the condition that the sufficiency score falls short of a pre-determined threshold value, supplemental content can be posted in the community chat in supplement to the expected response. In this way, despite an occurrence of an errant condition in the answering of a posted question in a community forum, a remedial measure can be undertaken so as to mitigate the consequence of the errant condition.

### BACKGROUND OF THE INVENTION

### Field of the Invention

The present invention relates to the technical field of question and answer processing in a community forum.

### Description of the Related Art

Society has become quite complex in the modern era. The lives of individuals span many decades and the environment in which one lives includes many facets. These many facets continuously provoke the desire in the individual to understand, to comprehend, to learn. However, the body of knowledge already understood by any one person pales in comparison to the knowledge sought which even further pales in comparison to what could be understood. Prior to the age of the Internet, one found knowledge in only three setting—the formalized academic setting, the informal inquisition of peers with or without the sought-after knowledge, and self-study, typically capitalizing on the resources of the library. The age of the Internet and more specifically, the search engine, however, has resulted in an eclipsing of the role of formal study with the published content of one's peers, known or unknown.

The search engine, a late 20th century tool of self-service learning, permits one to recall from the global repository of digitally published thought, documents of textually direct relevance to one or more keywords provided to the search engine. More advanced search engines of the 21st century permit a less direct searching of the corpus of digitally indexed content by inferring keywords from natural language questions submitted to the search engine. But in all instances, as a self-service tool, one is left with the burden of formulating an effective collection of search terms for submission to the search engine so as to achieve a likelihood of receiving relevant results. Moreover, it remains incumbent upon the individual to interpret the search results in a meaningful way, and to discriminate between reliable sources of data and those providing misinformation, whether intentional or unintentional. In many cases, the interpretation of search results only presents new questions requiring further searching.

The solitary nature of the search engine, in consequence, often is complemented with a community forum. In a community forum, different members exchange information and in response to the prompts of its membership, propose potential answers. This crowdsourcing of knowledge, while lacking the breadth of a search engine able to produce a high-volume result set, tends to produce a higher quality of result set than the search engine in so far as those participating in a community forum inherently enjoy a narrow focus upon the topicality of the knowledge sought by those participating in the forum. This expertise of the community forum, when available for a specific inquiry, produces a more relevant set of results to the inquiry much more quickly than the iterative and solitary inquisition of a search engine, and in many cases, in a synchronous or near synchronous and thus interactive manner so as to provide a more satisfactory experience for the inquisitor.

However, in many instances, within the setting of the community forum, a question posed to the community may not be answered satisfactorily. For instance, for a question for which time is of the essence, a delayed answer may not be sufficient. As well, sometimes an answer provided by a community member in response to a question posed by another community member may lack civility in tone or content, or the proffered answer may not be responsive in so far as the proffered answer may not address the question posed. Finally, in many instances, the ultimate risk of crowdsourced knowledge—false teachings—remains and without a large volume of responsive content for posed question, a response incorporating untrue information may receive undue weight by the inquisitor. Yet, the foregoing risks remain tolerated owing to the wider value of the community forum as a humanistic support mechanism in the digital era.

### BRIEF SUMMARY OF THE INVENTION

Embodiments of the present invention address technical deficiencies of the art in respect to question and answer processing in a digital community forum. To that end, embodiments of the present invention provide for a novel and non-obvious method for an automated facilitator for question and answer processing in a digital community forum. Embodiments of the present invention also provide for a novel and non-obvious computing device adapted to perform the foregoing method. Finally, embodiments of the present invention provide for a novel and non-obvious data processing system incorporating the foregoing device in order to perform the foregoing method.

In one embodiment of the invention, a method for automated third-party facilitation in a community chat includes monitoring an exchange of an inquisitor post in a community chat and an expected response to the inquisitor post. The method additionally includes processing the expected response to compute a sufficiency score measuring a sufficiency of the expected response to a question present in the inquisitor post. Thereafter, on the condition that the sufficiency score falls short of a pre-determined threshold value, supplemental content can be posted in the community chat in supplement to the expected response. In this way, despite an occurrence of an errant condition in the answering of a posted question in a community forum, a remedial measure can be undertaken so as to mitigate the consequence of the errant condition.

In one aspect of the embodiment, the sufficiency score can be computed based upon a presence of one or more keywords indicative of a failure to answer the question. In another aspect of the embodiment, the sufficiency score can be computed at least in part based upon a time of day when the inquisitor post had been submitted to the collaborative chat such that the supplemental content is a prompt to expect a delay in receiving the expected response to the inquisitor post owing to the time of day. In yet another aspect of the embodiment, the response as actually received may include text referring to external content, but lacking any activatable linkage to the external content, such that the supplemental content includes a hyperlink to the external content.

More broadly, as one option, the supplemental content is a set of one or more hyperlinks to external third party content which can be viewed as relevant to the question. The external third party content may include, by way of example, audio content, video content, or other rich formats. The supplemental content also may include text stored in a data store for the collaborative chat and that has been mapped to one or more keywords in the inquisitor post. To that end, in a specific aspect of the embodiment, the text stored in the data store may include one or more posts previously provided in previously conducted and now terminated collaborative chats as answers to corresponding questions determined to be semantically similar to the question of the inquisitor post. The supplemental content yet further can include a hyperlink to a community forum pertaining to a topic semantically including a context of the inquisitor post.

In another embodiment, of the invention, a data processing system can be adapted for automated third-party facilitation in a community chat. The system includes a host computing platform of one or more computers, each with memory and one or more processing units including one or more processing cores. The system also includes a chat facilitation module. The module includes computer program instructions enabled while executing in the memory of at least one of the processing units of the host computing platform to monitor an exchange of an inquisitor post in a community chat and an expected response to the inquisitor post or a lack of a timely, actual response to the inquisitor post, to process the expected response to compute a sufficiency score measuring a sufficiency of the response to a question present in the inquisitor post or the lack of a timely response to the inquisitor post, and, in the event that the sufficiency score falls short of a pre-determined threshold value, to post supplemental content in the community chat supplementing the response.

In even yet another embodiment of the invention, a computing device includes a non-transitory computer readable storage medium having program instructions stored therein. The instructions are executable by at least one processing core of a processing unit so as to cause the processing unit to perform a method for automated third-party facilitation in a community chat. To wit, the method includes monitoring an exchange of an inquisitor post in a community chat and a response to the inquisitor post or a delay in receiving a response to the inquisitor post, processing the response (or lack thereof) to compute a sufficiency score measuring a sufficiency of the response to a question present in the inquisitor post and, on the condition that the sufficiency score falls short of a pre-determined threshold value, posting supplemental content in the community chat supplementing the response.

In this way, the technical deficiencies of the question and answer management in a community forum are overcome owing to assurance that answers to a question posted in the community forum are provided in a timely manner and in a way most likely to convey relevant and accurate information to the inquisitor. In those circumstances where warranted, the supplemental content provided in addition to the answer serves to buttress the knowledge imported by the answer upon the inquisitor without requiring the inquisitor to post additional questions to the community forum. The result is the large scale reduction in resource utilization by avoiding unnecessary message exchanges between parties to the community forum.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration reflecting different aspects of a process of for the automated facilitation in question and answer processing within a digital community forum;

FIG. 2 is a block diagram depicting a data processing system adapted to perform one of the aspects of the process of FIG. 1; and,

FIG. 3 is a flow chart illustrating one of the aspects of the process of FIG. 1.

### DETAILED DESCRIPTION OF THE INVENTION

Embodiments of the invention provide for the automated facilitation in question and answer processing within a digital community forum. In accordance with an embodiment of the invention, an inquisitive posting is received from over a computer communications network into a digitally stored thread of a community forum. The thread is then monitored for a responsive posting to the inquisitive posting in the thread. The responsive posting, or a delay in receiving a responsive posting, is then processed to compute a sufficiency score. For instance, the sufficiency score can be computed based upon the relatedness of one or more words in the responsive posting to one or more words of the inquisitive posting, or to a topic computed based upon the inquisitive posting, or whether or not a clickable reference to external material is present in the responsive posting. Alternatively, the sufficiency score can be computed based upon an elapsed time measured before the responsive posting is received in the thread, if received at all.

To the extent that the sufficiency score crosses a threshold value, a supplemental posting is inserted into the thread incorporating supplemental content such as content retrieved from an index based upon one or more keywords in the inquisitive posting, content retrieved from an index based upon a topic determined from one or more keywords in the inquisitive posting, or an activatable link to external content either referenced in the responsive posting, or included in an index corresponding to one or more keywords in the responsive posting or corresponding to the topic of the inquisitive posting. To the extent that an unacceptable delay has been measured in receiving a responsive posting, the supplemental posting can be a message acknowledging the unexpected delay and encouraging patience.

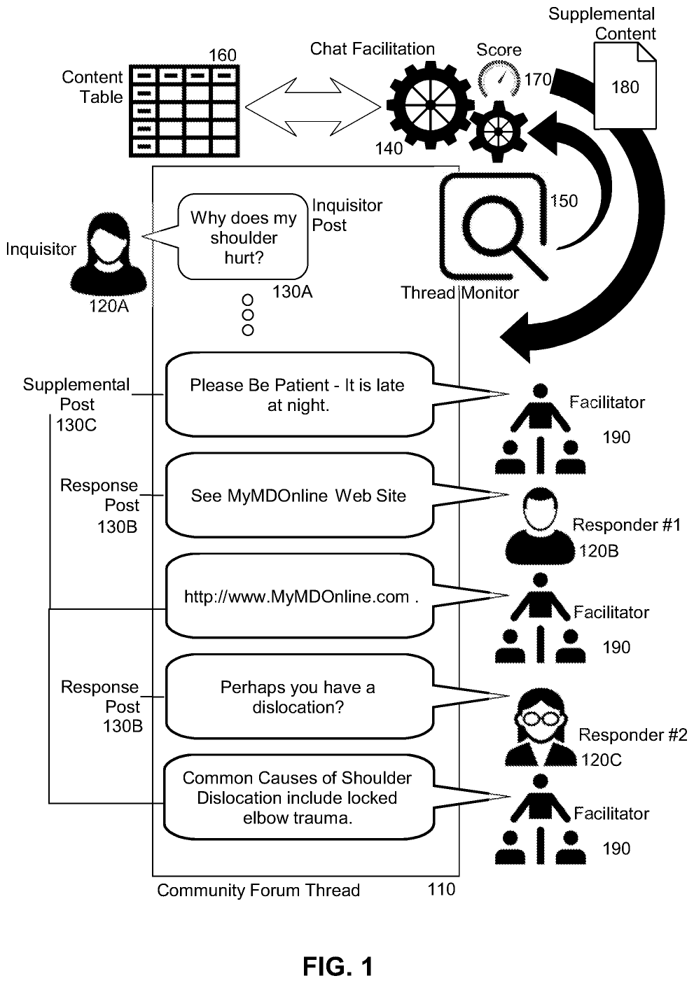

In illustration of one aspect of the embodiment, FIG. 1 pictorially shows a process of automated facilitation in question and answer processing within a digital community forum. As shown in FIG. 1, a community forum thread of discussion 110 hosts interactive postings 130A, 130B, 130C amongst different participants 120A, 120B, 120C to the thread 110. The postings 130A, 130B, 130C include both inquisitive postings 130A and responsive postings 130B to the inquisitive postings 130A by respectively different participants 120B, 120C. A thread monitor 150 acting as part of chat facilitation logic 140 monitors both the inquisitive postings 130A and responsive postings 130B.

Specifically, in one aspect of the embodiment, the thread monitor 150 detects an inquisitive posting 130A, for instance, by parsing a new one of the postings 130A, 130B to the thread 110, and then natural language processing the new one of the postings 130A, 130B in order to assess a topic. For example, to the extent that it is determined that the assessed topic represents a new topic, which differs from an existing topic for existing ones of the postings 130A, 130B in the thread 110 then the new one of the postings 130A, 130B will be considered an inquisitive posting 130A. Likewise, to the extent that punctuation in the new one or natural language analysis of the postings 130A, 130B indicates a question, then the new one of the postings 130A, 130B will be considered an inquisitive posting 130A. On the other hand, to the extent that the assessed topic does not differ from an existing topic for postings in the thread 110, the new one of the postings 130A, 130B can be considered a responsive posting 130B.

Once the thread monitor 150 has detected the inquisitive posting 130A, a sufficiency of a subsequent responsive posting 130B, if any, can be determined in the chat facilitation logic 140. Specifically, the chat facilitation logic 140 parses the content of each of the responsive postings 130B and computes a sufficiency score 170 for the content. The sufficiency score 170 indicates a relatedness and thoroughness of the responsive posting 130B to the inquisitive posting 130A. For instance, to the extent that the responsive posting 130B includes references to Web accessible content while also including a hyperlink to reach the Web accessible content, a higher value for the sufficiency score 170 is computed whereas in the opposite instance, a lower value for the sufficiency score 170 is computed.

As well, to the extent that a technical term is used in the responsive posting 130B without further elaboration, a lower sufficiency score 170 is computed whereas a higher value for the sufficiency score 170 is computed when a technical term is accompanied within the responsive posting 130B by explanatory terms. In this regard, a sophistication determination can be applied to the inquisitive posting 130A based upon a degree of complexity of words used in the inquisitive posting 130A, with more complex words indicating a higher degree of sophistication and more simple words indicating a lower degree of sophistication, the complexity of the words being referenced in a dictionary of words assigning complexity values to words. Thereafter, the sufficiency score 170 can be weighted lower for an inquisitive posting of higher sophistication and weighted higher for an inquisitive posting of lower sophistication.

As another example, a lower value for the sufficiency score 170 is assigned to a responsive posting 130B when the topicality determined for the responsive posting 130B differs from the topicality of the inquisitive posting 130A. As even yet another example, to the extent that a textual reference is included as a source of information within the responsive posting 130B that correlates to a known source of misinformation is listed within a table of suspect sources, a lower value for the sufficiency score 170 is assigned. Finally, to the extent that no responsive posting 130B is received within a threshold time period following the inquisitive posting 130A, a low value for the sufficiency score 170 is established.

To the extent that the chat facilitation logic 140 determines that the sufficiency score 170 does not cross a threshold value, no further action is taken and the responsive posting 130B remains in the thread 110 without supplement. But, to the extent that the chat facilitation logic 140 determines that the sufficiency score 170 has crossed the threshold value, the chat facilitation logic 140 resolves to supplement the content in the thread 110 with supplemental content 180.

In this regard, to the extent that the chat facilitation logic 140 detects a reference to a Web site in a responsive posting 130B, the chat facilitation logic 140 can query a content table 160 in order to locate a uniform resource locator (URL) for the Web site. The chat facilitation logic 140 then can insert the URL as supplemental content 180 into a supplemental posting 130C on behalf of a facilitator participant 190 to the thread 110. Likewise, the chat facilitation logic 140 can query the content table 160 with a keyword located within either the inquisitive posting 130A or within the responsive posting 130B in order to retrieve content related to the keyword that has been stored either internally or that is accessible at a Web site over the global Internet. In either instance, the supplemental content 180 is then provided as a supplemental posting 130C on behalf of the facilitator participant 190 to the thread 110.

As another example, the inquisitive posting 130A can be parsed into keywords which can be cross-referenced in an index to one or more expected response terms. The responsive posting 130B also can be parsed into keywords and compared to the expected response terms. In one embodiment, ones of the expected response terms not present in the parsed keywords of the responsive posting 130B are then used as query terms in a data store of indexed entries for use in constructing the supplemental content to be provided as a supplemental posting 130C. In another embodiment, the determination that ones of the expected response terms is not present in the parsed keywords of the responsive posting 130B results in providing the complete model answer in the supplemental content 180 to the inquisitive posting. Alternatively, a topic is determined from the inquisitive posting 130A and a model answer retrieved corresponding to the topic. A semantic analysis is then performed upon the text of the responsive posting 130B and compared to the model answer. To the extent that the model answer is determined to differ from the semantic analysis of the responsive posting 130B, the model answer is supplied as the supplemental content 180.

As yet a further example, a sentiment analysis is determined for the responsive posting 130B. To the extent that a hostile or aggressive sentiment is determined for the responsive posting 130B, the keywords of the inquisitive posting 130A are used as keyword queries to pre-fabricated content for use in a supplemental posting 130C.

Optionally, to the extent that no responsive posting 130B has been received within a threshold period of time, the chat facilitation logic 140 provides a request for patience as the supplemental content 180. For example, upon determining that no responsive posting 130B has been received within a threshold period of time thus giving rise to a low value for the sufficiency score 170, if it is additionally determined that the time of the inquisitive posting 130A had been late in the evening, the supplemental content 180 can include an indication that a delay in receiving the responsive posting 130B is not unexpected given the time of day. Similarly, upon determining that no responsive posting 130B has been received within a threshold period of time thus giving rise to a low value for the sufficiency score 170, if it is additionally determined that the date of the inquisitive posting 130A had been a holiday, the supplemental content 180 can include an indication that a delay in receiving the responsive posting 130B is not unexpected given the holiday.

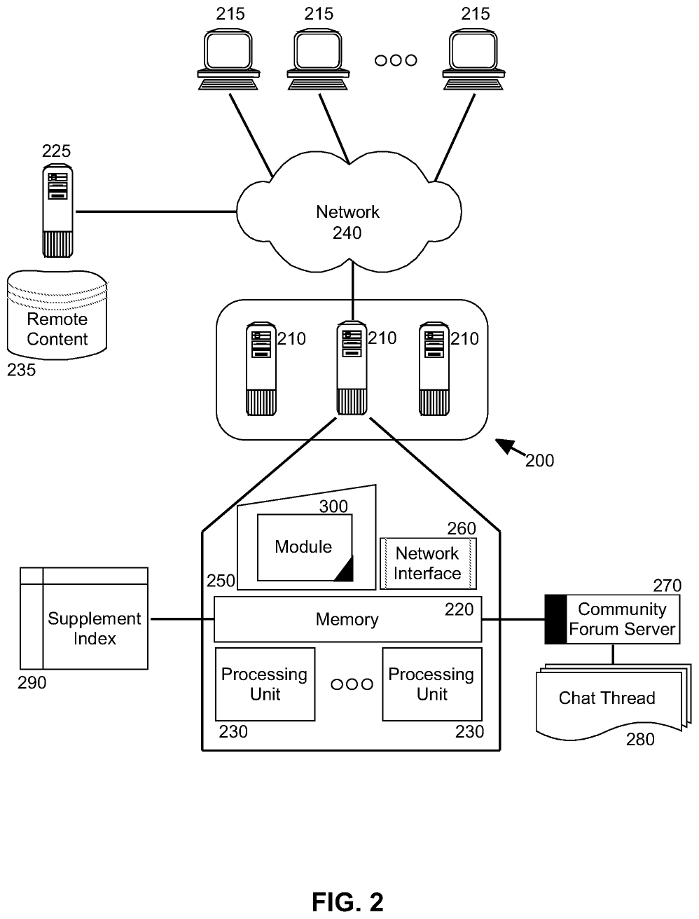

Aspects of the process described in connection with FIG. 1 can be implemented within a data processing system. In further illustration, FIG. 2 schematically shows a data processing system adapted to perform automated facilitation in question and answer processing within a digital community forum. In the data processing system illustrated in FIG. 2, a host computing platform 200 is provided. The host computing platform 200 includes one or more computers 210, each with memory 220 and one or more processing units 230. The computers 210 of the host computing platform (only a single computer shown for the purpose of illustrative simplicity) can be co-located within one another and in communication with one another over a local area network, or over a data communications bus, or the computers can be remotely disposed from one another and in communication with one another through network interface 260 over a data communications network 240.

A community forum server 270 executes in the memory 220 by one or more of the processing units 230. The community forum server 270 creates and maintains multiple different chat threads 280 in which posts are submitted by different participants to the chat threads 280 from over the data communications network 240. In this regard, the different participants submit the posts through respective client devices 215, which may include for instance both personal computing devices and also mobile computing devices, such that one of the participants submits to the community forum server 270 an inquisitor posting for placement in a particular one of the chat threads 280 and others of the participants submit to the community forum server 270 responsive postings to the inquisitor posting for placement in the particular one of the chat threads 280.

Notably, a computing device 250 including a non-transitory computer readable storage medium can be included with the data processing system 200 and accessed by the processing units 230 of one or more of the computers 210. The computing device stores 250 thereon or retains therein a program module 300 that includes computer program instructions which when executed by one or more of the processing units 230, performs a programmatically executable process for automated facilitation in question and answer processing within a digital community forum. Specifically, the program instructions during execution detect an inquisitor posting in one of the chat threads 280. Thereafter, the program instructions assess a sufficiency of a responsive posting to the inquisitory posting in the one of the chat threads 280 and compute a sufficiency score for the responsive posting.

To the extent the responsive posting has a sufficiency score computed to be below a threshold value, the program instructions access a supplement index 290 in the memory 220 with at least one keyword extracted from either the responsive posting or the inquisitor posting in order to retrieve supplemental content. Alternatively, a reference to supplemental content disposed in a remote content store 235 managed by remote server 225 is retrieved from the supplement index 290, such as a reference to a posting in another of the chat threads 280, or Web content published by a Web server from over the data communications network 240. Finally, the program instructions insert the supplemental content in a facilitated posting in the one of the chat threads 280 in supplement to the responsive posting.

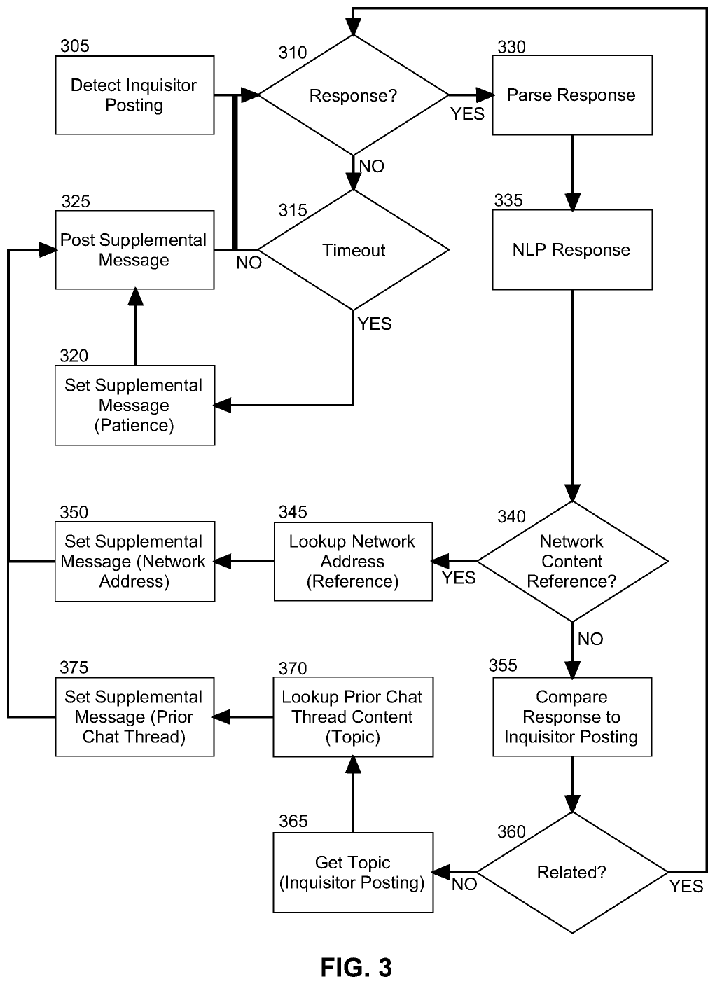

In further illustration of an exemplary operation of the module, FIG. 3 is a flow chart illustrating one of the aspects of the process of FIG. 1. Beginning in block 305, an inquisitor posting from an inquisitor participant in a chat thread of a community forum is detected in the chat thread. In decision block 310, it is determined whether or not an expected responsive posting has been received in connection with the inquisitor posting. If not, in decision block 315 it is further determined if a threshold amount of time has elapsed since the inquisitor posting had been posted to the chat thread. If so, in block 320 a supplemental message is created prompting the inquisitor to not lose patience and in block 325 the message is posted as a supplemental posting in the chat thread. Optionally, the content of the inquisitor posting can be parsed to detect words associated with urgency and upon detecting words associated with urgency, the threshold amount of time can be reduced, or in the absence of words associated with urgency, no supplemental message need be posted in block 320 notwithstanding the lapse of the threshold amount of time.

In decision block 310, if it is determined that a responsive posting as expected has been received in connection with the inquisitor posting, in block 330 the responsive posting is parsed and inserted into one or more sentences, each being structured into a graph, and in block 335, each graph is natural language processed in order to determine the sufficiency of the responsive posting in respect to the inquisitor posting. Specifically, in decision block 340, it can be determined within one of the graphs whether or not network content has been referenced. If so, in block 345 a network address can be retrieved from a data store in correspondence to the referenced network content. Then, in block 350, a supplemental message is formulated incorporating the network address and posted as a supplemental posting to the chat thread in block 325.

In decision block 340, if no network content is referenced in the graphs, in block 355, the contents of the graphs are compared to the content of the inquisitor posting and in decision block 360, it is determined if a topical relationship exists between the inquisitor posting and each of the graphs. If not, in block 365, the topic for the inquisitor posting is retrieved and in block 370, topically pertinent content for the topic is located for instance in reference to result text retrieved by a query to a remote content store using the topic as a keyword, or in reference to result text retrieved by a query using the topic as a keyword to a local content store of previous chat threads. Then, in block 375 a supplemental message is formulated incorporating the topically pertinent content in the different chat thread. Finally, in block 325 the supplemental message is posted to the chat thread as a supplemental posting.

Of import, the foregoing flowchart and block diagram referred to herein illustrate the architecture, functionality, and operation of possible implementations of systems, methods, and computing devices according to various embodiments of the present invention. In this regard, each block in the flowchart or block diagrams may represent a module, segment, or portion of instructions, which includes one or more executable instructions for implementing the specified logical function or functions. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

More specifically, the present invention may be embodied as a programmatically executable process. As well, the present invention may be embodied within a computing device upon which programmatic instructions are stored and from which the programmatic instructions are enabled to be loaded into memory of a data processing system and executed therefrom in order to perform the foregoing programmatically executable process. Even further, the present invention may be embodied within a data processing system adapted to load the programmatic instructions from a computing device and to then execute the programmatic instructions in order to perform the foregoing programmatically executable process.

To that end, the computing device is a non-transitory computer readable storage medium or media retaining therein or storing thereon computer readable program instructions. These instructions, when executed from memory by one or more processing units of a data processing system, cause the processing units to perform different programmatic processes exemplary of different aspects of the programmatically executable process. In this regard, the processing units each include an instruction execution device such as a central processing unit or “CPU” of a computer. One or more computers may be included within the data processing system. Of note, while the CPU can be a single core CPU, it will be understood that multiple CPU cores can operate within the CPU and in either instance, the instructions are directly loaded from memory into one or more of the cores of one or more of the CPUs for execution.

Aside from the direct loading of the instructions from memory for execution by one or more cores of a CPU or multiple CPUs, the computer readable program instructions described herein alternatively can be retrieved from over a computer communications network into the memory of a computer of the data processing system for execution therein. As well, only a portion of the program instructions may be retrieved into the memory from over the computer communications network, while other portions may be loaded from persistent storage of the computer. Even further, only a portion of the program instructions may execute by one or more processing cores of one or more CPUs of one of the computers of the data processing system, while other portions may cooperatively execute within a different computer of the data processing system that is either co-located with the computer or positioned remotely from the computer over the computer communications network with results of the computing by both computers shared therebetween.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for automated third-party facilitation in a community chat, the method comprising:
monitoring a submission of an inquisitor post in a community chat and an expected response to the inquisitor post;
processing the expected response to compute a sufficiency score measuring a sufficiency of the expected response to a question present in the inquisitor post; and,
on condition that the sufficiency score falls short of a pre-determined threshold value, posting supplemental content in the community chat supplementing the expected response.

2. The method of claim 1, wherein the sufficiency score is computed based upon a presence of one or more keywords indicative of a failure to answer the question.

3. The method of claim 1, wherein the supplemental content is a set of one or more hyperlinks to external third party content.

4. The method of claim 1, wherein the supplemental content is text stored in a data store for the collaborative chat that is mapped to one or more keywords in the inquisitor post.

5. The method of claim 4, wherein the text stored in the data store includes one or more posts previously provided in previously conducted and now terminated collaborative chats as answers to corresponding questions determined to be semantically similar to the question of the inquisitor post.

6. The method of claim 1, wherein the supplemental content is a hyperlink to a community forum pertaining to a topic semantically including a context of the inquisitor post.

7. The method of claim 1, wherein the sufficiency score is computed at least in part based upon a time of day when the inquisitor post is submitted to the collaborative chat.

8. The method of claim 1, wherein the expected response includes text referring to external content and the supplemental content is a hyperlink to the external content.

9. A data processing system adapted for automated third-party facilitation in a community chat, the system comprising:
a host computing platform comprising one or more computers, each with memory and one or more processing units including one or more processing cores; and,
a chat facilitation module comprising computer program instructions enabled while executing in the memory of at least one of the processing units of the host computing platform to perform:
monitoring an exchange of an inquisitor post in a community chat and an expected response to the inquisitor post;
processing the response to compute a sufficiency score measuring a sufficiency of the expected response to a question present in the inquisitor post; and,
on condition that the sufficiency score falls short of a pre-determined threshold value, posting supplemental content in the community chat supplementing the expected response.

10. The system of claim 9, wherein the sufficiency score is computed based upon a presence of one or more keywords indicative of a failure to answer the question.

11. The system of claim 9, wherein the supplemental content is a set of one or more hyperlinks to external third party content.

12. The system of claim 9, wherein the supplemental content is text stored in a data store for the collaborative chat that is mapped to one or more keywords in the inquisitor post.

13. The system of claim 12, wherein the text stored in the data store includes one or more posts previously provided in previously conducted and now terminated collaborative chats as answers to corresponding questions determined to be semantically similar to the question of the inquisitor post.

14. The system of claim 9, wherein the supplemental content is a hyperlink to a community forum pertaining to a topic semantically including a context of the inquisitor post.

15. The system of claim 9, wherein the sufficiency score is computed at least in part based upon a time of day when the inquisitor post is submitted to the collaborative chat and the supplemental content is a prompt to expect a delay in receiving the expected response to the inquisitor post owing to the time of day.

16. The system of claim 9, wherein the expected response includes text referring to external content and the supplemental content is a hyperlink to the external content.

17. A computing device comprising a non-transitory computer readable storage medium having program instructions stored therein, the instructions being executable by at least one processing core of a processing unit to cause the processing unit to perform a method for automated third-party facilitation in a community chat, the method including:
monitoring an exchange of an inquisitor post in a community chat and an expected response to the inquisitor post;
processing the expected response to compute a sufficiency score measuring a sufficiency of the expected response to a question present in the inquisitor post; and,
on condition that the sufficiency score falls short of a pre-determined threshold value, posting supplemental content in the community chat supplementing the expected response.

18. The device of claim 17, wherein the sufficiency score is computed based upon a presence of one or more keywords indicative of a failure to answer the question.

19. The device of claim 17, wherein the supplemental content is text stored in a data store for the collaborative chat that is mapped to one or more keywords in the inquisitor post.

20. The device of claim 19, wherein the text stored in the data store includes one or more posts previously provided in previously conducted and now terminated collaborative chats as answers to corresponding questions determined to be semantically similar to the question of the inquisitor post.
